(19) United States
(12) Plant Patent Application Publication
(10) Pub. No.: US 2015/0305217 P1
(43) Pub. Date: Oct. 22, 2015

(54) Petunia plant named 'KLEPH13239'

(50) ;

(71) Applicant: Klemm+Sohn GmbH & Co. KG, Stuttgart (DE)

(72) Inventors: Nils Klemm, Stuttgart (DE); Antonella Capo, Latina (IT)

(73) Assignee: Klemm+Sohn GmbH & Co. KG, Stuttgart (DE)

(21) Appl. No.: 14/120,064

(22) Filed: Apr. 21, 2014

(51) Int. Cl. A01H 5/00

(52) U.S. Cl. PLT/356.1

(57) ABSTRACT

A new Petunia plant particularly distinguished by orange flowers having lighter throats and weak veins, a medium-compact form, and an upright-mounding plant habit, is disclosed.

### GENUS AND SPECIES

Petunia hybrida

### VARIETY DENOMINATION

‘KLEPH13239’

### BACKGROUND OF THE NEW PLANT

The present invention comprises a new and distinct variety of Petunia, botanically known as Petunia hybrida, and hereinafter referred to by the variety name ‘KLEPH13239’. ‘KLEPH13239’ originated from a cross-pollination conducted in July 2010 in Latina, Italy between the female Petunia variety ‘Surprise Orange Twist’ (patent status unknown) and the male Petunia variety SUNSPUN ‘Red’ (patent status unknown).

The seeds from the cross were sown and plants were grown in a greenhouse for evaluation, where an individual plant designated ‘KLEPH13239’ was selected from the group in April 2011 of plants in Latina, Italy is this correct, having orange flowers with weak veins. In April 2011, ‘KLEPH13239’ was first vegetatively propagated by terminal tip cuttings and tissue culture. ‘KLEPH13239’ was found to reproduce true to type in successive generations of asexual propagation via tissue culture and terminal tip cuttings.

### SUMMARY

The following are the most outstanding and distinguishing characteristics of this new variety when grown under normal horticultural practices in Stuttgart, Germany and Latina, Italy.

1. Orange flowers having lighter throats and weak veins;

2. A medium-compact form; and

3. An upright-mounding plant habit.

### DESCRIPTION OF THE PHOTOGRAPHS

This new Petunia plant is illustrated by the accompanying photographs of plants grown in a greenhouse in Stuttgart, Germany. The colors shown are as true as can be reasonably obtained by conventional photographic procedures.

FIG. 1 shows a photograph taken in July 2013 of a 5-month-old plant grown in a basket.

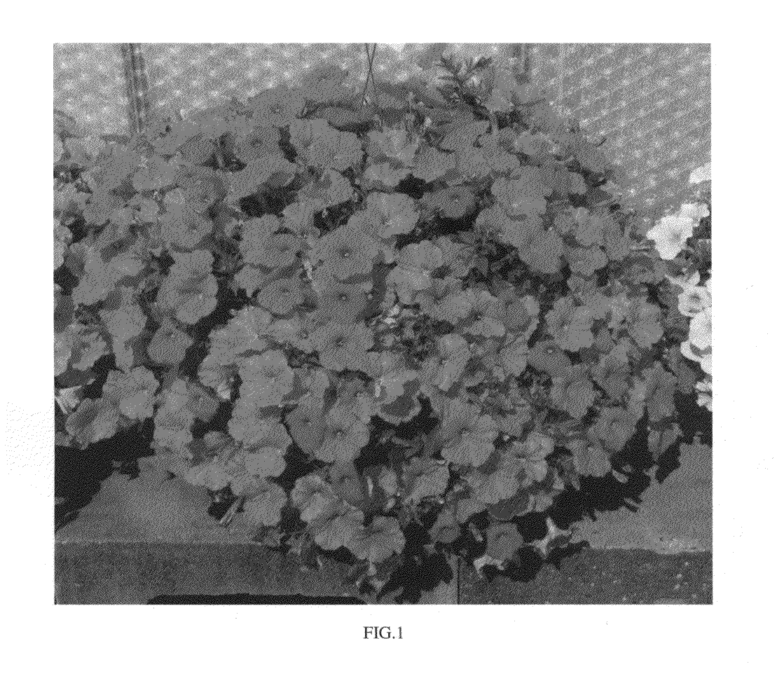

FIG. 2 shows a photograph taken in May 2013 of a 3-month-old plant grown in a pot.

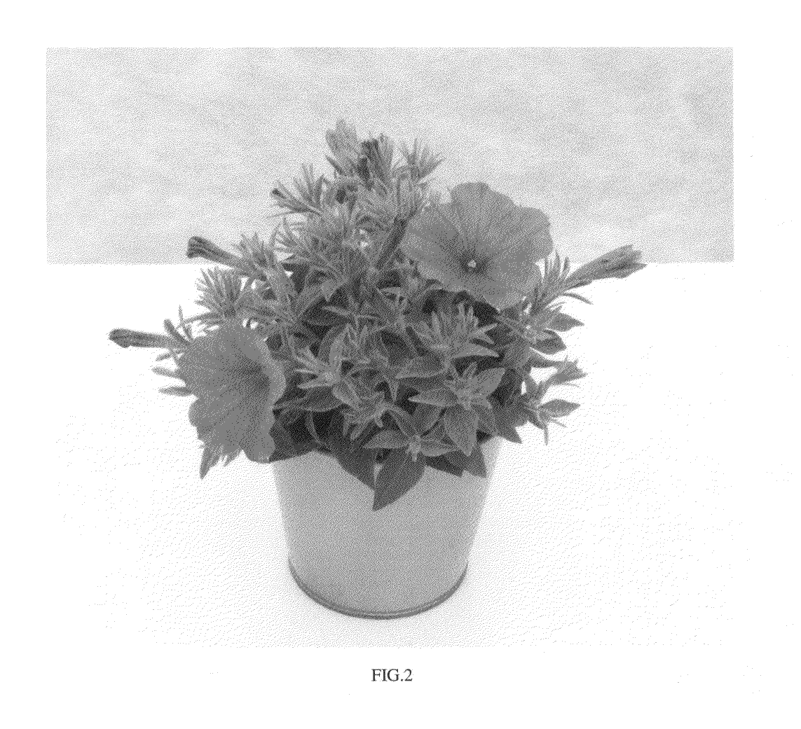

### DESCRIPTION OF THE NEW VARIETY

The following detailed descriptions set forth the distinctive characteristics of ‘KLEPH13239’. The data which define these characteristics were collected from asexual reproductions carried out in Latina, Italy. Data was collected on plants grown 14-weeks from cultivation from potting in a plastic greenhouse in Latina, Italy in March 2014. Color references are to The R.H.S. Colour Chart of The Royal Horticultural Society of London (R.H.S.), 5^{th} edition (2007).
Classification:
   Family.—Solanaceae.
   Botanical.—Petunia hybrid.
   Common.—Petunia.
   Designation.—‘KLEPH13239’.
Parentage:
   Female parent.—The female Petunia variety ‘Surprise Orange Twist’ (patent status unknown).
   Male parent.—The male Petunia variety SUNSPUN ‘Red’ (patent status unknown).
Plant:
   Form.—Medium-compact.
   Habit.—Upright-mounding.
   Height (from top of soil).13 About 12.0 cm.
   Width (horizontal plant diameter).—About 24.0 cm.
   Propagation.—Terminal tip cuttings and tissue culture.
   Time to produce a finished flowering plant.—14 to 15 weeks in the winter and 7 weeks in the spring.
   Time to initiate and develop roots.—2 to 3 weeks.
   Root description.—Moderate density, moderate branching, white roots.
Stems:
   Average number (basal).—About 18.
   Length of basal branches (from the base of the stem to the tip).—About 12.0 cm.
   Internode length.—1.0 cm to 2.5 cm.
   Diameter of branches (from midpoint).—0.3 cm.
   Stem color.—RHS 144B.
   Anthocyanin.—Absent.
   Texture.—Pubescent.
Leaves:
   Arrangement.—Alternate at the base of the stems, opposite on the flower stems.
   Length.—3.0 cm to 3.5 cm.
   Width.—1.5 cm to 2.0 cm.
   Shape.—Elliptic.
   Apex.—Acute.
   Base.—Attenuate.
   Margin.—Entire.
   Immature and mature leaf color.—Upper surface: RHS N137D Lower surface: RHS 138B.
   Texture (both upper and lower surfaces).—Pubescent.
   Venation pattern.—Arcuate.
   Venation color.—Upper surface: RHS 137B Lower surface: RHS 138B.
   Petioles.—Length: 0.3 cm Diameter: 0.2 cm Color: RHS 137B Texture: Pubescent.
Flower buds:
   Shape.—Irregular oblong.
   Length.—About 3.0 cm.
   Diameter.—0.3 cm.
   Color at tight bud.—RHS 176A.
   Texture.—Slightly pubescent.
Inflorescence:
   Blooming habit (flowering season).—Permanent flowering during summer time.
   Inflorescence type.—Cymose.
   Number of flowers per node.—1.
   Lastingness of individual blooms on the plant.—7 to 10 days.
   Fragrance.—Absent.
   Flowers.—Arrangement: Composed of 5 petals fused at the base Shape: Funnel-form Diameter (flower face): 6.0 cm Depth (total length of flower): 6.0 cm Throat/Funnel: Length: 2.0 cm Diameter (at opening): 1.3 cm Texture (both inner and outer surfaces): Smooth, glabrous Petals: Apex: Mucronate Base: Fused Margin: Entire Strength of waviness: Weak Degree of lobation: Medium-weak Calyx arrangement: Actinomorphic, composed of 5 sepals Sepals: Color: Upper surface: RHS N137D Lower surface: RHS 138B Length: 2.0 cm Width: 0.4 cm Shape: Linear Apex: Acute Base: Attenuate Margin: Entire Texture (both upper and lower surfaces): Pubescent.
   Pedicels.—Color: RHS 144A Length: 3.5 cm Diameter: 0.15 cm Texture: Pubescent.
Reproductive organs:
   Stamens.—Quantity: 5 Shape: Needle with elliptic head Filament: Length: 1.0 cm to 1.2 cm Diameter: About 0.02 cm Color: RHS 36C Anther: Shape: Elliptic Color: RHS 27B Length: 0.3 cm Diameter: 0.2 cm Pollen: Color: RHS 157B Amount: Moderate.
   Pistils.—Quantity per flower: 1 Length: 1.8 cm Stigma color: RHS 157A Style color: RHS 160D.
   Fruit and seed set.—No fruit and seed set observed.
   Disease and pest/insect resistance.—No disease and pest/insect resistance observed.

### COMPARISON WITH PARENTAL LINES AND KNOWN VARIETY

‘KLEPH13239’ is a distinct variety of Petunia and is distinguished from its parents as described in Table 1.

TABLE 1
Comparison with Parental Lines

| | | Female Parent | Male Parent |
|---|---|---|---|
| | | ‘Surprise | SUNSPUN |
| Characteristic | ‘KLEPH13239’ | Orange Twist’ | ‘Red’ |
| Flower color | Lighter orange | Brilliant orange | Red |
| | with a light throat | with veins and | |
| | and weak veins | a dark throat | |

‘KLEPH13239’ is a distinct variety of Petunia and is most similar to the commercial Petunia variety SUNSPUN ‘Red’ (patent status unknown). Differences between the varieties are described in Table 2.

TABLE 2
Comparison with Similar Variety

| Characteristic | ‘KLEPH13239’ | SUNSPUN ‘Red’ |
|---|---|---|
| Flower color | Orange with weak veins | Red |

We claim:

1. A new and distinct variety of Petunia plant designated ‘KLEPH13239’ as shown and described herein.
